(19) United States
(12) Patent Application Publication
(10) Pub. No.: US 2023/0060797 A1
(43) Pub. Date: Mar. 2, 2023

(54) THERAPEUTIC COMPOUND FOR NEURONAL CEROID LIPOFUSCINOSIS

(71) Applicant: JCR Pharmaceuticals Co., Ltd., Ashiya-shi (JP)

(72) Inventor: Mathias SCHMIDT, San Diego, CA (US)

(73) Assignee: JCR Pharmaceuticals Co., Ltd., Ashiya-shi (JP)

(21) Appl. No.: 17/465,478

(22) Filed: Sep. 2, 2021

(51) Int. Cl. A61K 31/4375; A61P 25/28

(52) U.S. Cl. CPC A61K 31/4375; A61P 25/28

(57) ABSTRACT

[Problem to be solved] To provide a therapeutic compound for neuronal ceroid lipofuscinosis. [Solution] Provided is a compound for treatment and/or prevention of neuronal ceroid lipofuscinosis represented by the following general formula [I]. In the general formula [I], R1 is a methyl group or a hydroxymethyl group, R2 is a methyl group or a hydroxymethyl group, R3 is one of a hydroxy group, a methoxy group, an ethoxy group, an isopropoxy group, a methoxyethoxy group, a methoxypropoxy group, or an ethoxypolopoxy group, and R4 is one of a hydroxy group, a methoxy group, an ethoxy group, an isopropoxy group, a methoxyethoxy group, a methoxypropoxy group, or an ethoxypolopoxy group. In particular, provided is a compound represented by the general formula [I] with R1 being a methyl group, R2 being a methyl group, R3 being a hydroxy group, and R4 being a methoxyethoxy group.

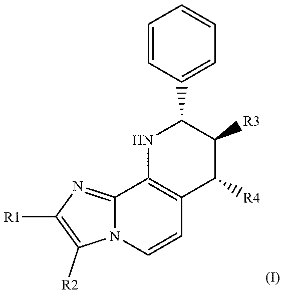

### TECHNICAL FIELD

The present invention relates to a compound for treatment of neuronal ceroid lipofuscinosis.

### TECHNICAL BACKGROUND

Neuronal ceroid lipofuscinosis (NCL) or Batten disease, which is a type of lysosomal disease, is a hereditary disease characterized by progressive neurodegeneration in which, due to mutations in any of multiple causative genes such as CLN1, CLN2, and CLN3, lipofuscin accumulates in intracellular lysosomes, causing blindness, muscle coordination ataxia, mental retardation or decreased mental function, emotional disturbance, seizures, muscle spasms, decreased muscle tone, movement disorders, and the like. It is classified into 14 types according to genes that show abnormalities.

Neuronal ceroid lipofuscinosis includes an infant type (Santa of Ori-Hartia disease), a late infant type (Jansky-Beershawski disease), a juvenile type (Spielmeier-Fogt disease), and an adult type (Koufus disease or Parry disease).

Enzyme replacement therapy using recombinant TPP1 (Cell liponase alpha, BioMarin Pharmaceuticals) for treatment of neuronal ceroid lipofuscinosis type 2 has recently been approved in the United States, Europe, and Japan and administration using an intracerebroventricular administration system has been performed (Patent Document 1). However, for other types of neuronal ceroid lipofuscinosis, currently, there is no effective treatment and only symptomatic treatment and supportive treatment are available.

### RELATED ART

### Patent Document

[Patent Document 1] International Publication No. 2016/182862.

### SUMMARY OF THE INVENTION

### Problems to be Solved by the Invention

An object of the present invention is to provide a therapeutic compound for neuronal ceroid lipofuscinosis.

### Means for Solving the Problems

In a research aimed at the above object, the present inventors have found that a compound according to the present invention can reduce lipofuscin accumulated in cells of a patient with neuronal ceroid lipofuscinosis, and thus accomplished the present invention. That is, the present invention includes the following.

1. A compound for treatment and/or prevention of neuronal ceroid lipofuscinosis represented by the following general formula [I] wherein in the general formula [I], R1 is a methyl group or a hydroxymethyl group, R2 is a methyl group or a hydroxymethyl group, R3 is one of a hydroxy group, a methoxy group, an ethoxy group, an isopropoxy group, a methoxyethoxy group, a methoxypropoxy group, or an ethoxypolopoxy group, and R4 is one of a hydroxy group, a methoxy group, an ethoxy group, an isopropoxy group, a methoxyethoxy group, a methoxypropoxy group, or an ethoxypolopoxy group.

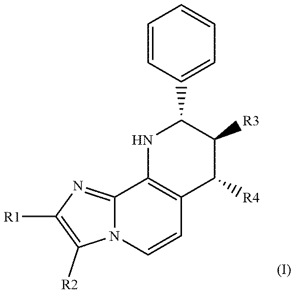

2. The compound according to 1 above, wherein, in the general formula [I], R1 is a methyl group.

3. The compound according to 1 above, wherein, in the general formula [I], R1 is a methyl group, and R2 is a methyl group.

4. The compound according to 1 above, wherein, in the general formula [I], R3 is a hydroxy group.

5. The compound according to 1 above, wherein, in the general formula [I], R3 is a hydroxy group, and R4 is a methoxyethoxy group.

6. The compound according to 1 above, wherein, in the general formula [I], R1 is a methyl group, R2 is a methyl group, R3 is a hydroxy group, and R4 is a methoxyethoxy group.

7. A pharmaceutical composition for treatment and/or prevention of neuronal ceroid lipofuscinosis, comprising a therapeutically effective amount of the compound according to 1 above or a pharmaceutically acceptable salt thereof.

8. The pharmaceutical composition according to 7 above characterized by that, when administered to a patient with neuronal ceroid lipofuscinosis, lipofuscin accumulated in the patient’s body is reduced.

9. The pharmaceutical composition according to 8 above, wherein the administration is subcutaneous, intravenous, intramuscular, parenteral, local, oral, transdermal, intraperitoneal, intraorbital, implantation, inhalation, intraarachnoid, intravesical, or intranasal administration.

10. The pharmaceutical composition according to 7 above, being in a dosage form selected from a group consisting of tablets, capsules, granules, powders, oral solutions, syrups, oral jelly, oral

tablets, oral sprays, oral semi-solid preparations, orally disintegrating tablets, parenteral emulsions, parenteral suspensions, parenteral solutions, effervescent tablets, mouthwash, injections, and inhalants.

11. The pharmaceutical composition according to 7 above, further comprising at least one pharmaceutically acceptable additive.

12. The pharmaceutical composition according to 11 above, wherein the at least one additive is selected from a group consisting of a flavoring agent, a foaming agent, a fragrance, an excipient, an isotonic agent, a surfactant, an emulsifier, a colorant, a buffer, zinc or its salt, a preservative, an antioxidant, a pH adjusting agent, a stabilizer, and a suspension agent or a solubilizing agent.

13. The pharmacological composition according to 7 above, being used together with enzyme replacement therapy for a patient with neuronal ceroid lipofuscinosis.

### Effect of the Invention

According to the present invention, a therapeutic compound for neuronal ceroid lipofuscinosis can be provided.

### Mode for Carrying Out the Invention

A compound according to the present invention can be represented by the following general formula [I].

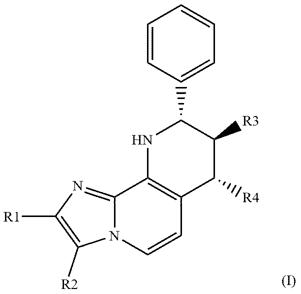

In the above general formula [I], R1 is not particularly limited, but is preferably a methyl group or a hydroxymethyl group, and more preferably a methyl group.

In the above general formula [I], R2 is not particularly limited, but is preferably a methyl group or a hydroxymethyl group, and more preferably a methyl group.

In the above general formula [I], R3 is not particularly limited, but is preferably a hydroxy group, a methoxy group, an ethoxy group, an isopropoxy group, a methoxyethoxy group or a methoxypropoxy group, more preferably a hydroxy group, a methoxy group or an ethoxy group, and even more preferably a hydroxy group.

In the above general formula [I], R4 is not particularly limited, but is preferably a hydroxy group, a methoxy group, an ethoxy group, an isopropoxy group, a methoxyethoxy group or a methoxypropoxy group, more preferably a methoxyethoxy group or a methoxypropoxy group, and even more preferably a methoxyethoxy group.

A substance represented by a chemical formula [ II], which is the above general formula [I] with R1 being a methyl group, R2 being a methyl group, R3 being a hydroxy group, and R4 being a methoxyethoxy group, is a compound known by the name Soraprazan or BYK61359.

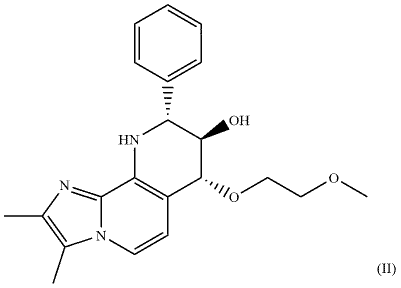

The compound according to the present invention can be used for treatment and/or prevention of neuronal ceroid lipofuscinosis. Neuronal ceroid lipofuscinosis is a disease caused by accumulation of lipofuscin in lysosomes of cells. The compound according to the present invention can remove the accumulated lipofuscin from the cells, and thus can be used for the treatment of neuronal ceroid lipofuscinosis. Further, the compound according to the present invention can inhibit new accumulation of lipofuscin in the lysosome of the cell, and thus can also be used for the prevention of neuronal ceroid lipofuscinosis.

Neuronal ceroid lipofuscinosis, also called Batten disease, is classified into multiple types according to genes that show abnormalities, including an infant type (Santa of Ori-Hartia disease), a late infant type (Jansky-Beershawski disease), a juvenile type (Spielmeier-Fogt disease), an adult type (Koufus disease or Parry disease), and the like. However, the term “neuronal ceroid lipofuscinosis” as used herein includes all types or disease names as long as it is a disease that develops physical symptoms due to accumulation of lipofuscin in lysosomes of cells that form the patient’s body tissue due to a congenital genetic abnormality. At least 14 mutations of variants in different genes are known to result in the excessive accumulation of lipofuscin in tissues and organs of somatic and central nervous parts of the body.

As used herein, the term “treatment” means ameliorating an existing medical condition, and the term “prevention” means reducing an expected serious medical condition and/or slowing progression of a disease.

The compound according to the present invention can be formulated as a pharmaceutical composition containing a therapeutically effective amount thereof for use in the treatment and/or prevention of all types of neuronal ceroid lipofuscinosis. In this case, the compound according to the present invention may be in a form of a pharmaceutically acceptable salt. The term “salt” refers to a compound in which cations and anions are ionically bonded, and includes a combination of cations and anions that are ionized in an aqueous solution state.

As used herein, the term “therapeutically effective amount” refers to an amount of a compound that is effective in alleviating a condition of the patient’s disease by administration of the compound to the patient. A therapeutic effect and toxicity of such a compound can be determined by standard pharmaceutical procedures in cell culture or laboratory animals. A dose is preferably in a range of circulating levels including ED50 with little or no toxicity. The dose is appropriately selected depending on a dosage form used, age and other patient conditions, a route of administration, and the like.

The pharmaceutically acceptable salt of the compound according to the present invention is not particularly limited, and is, for example, hydrochloride, hydrobromide, iohydrate, nitrate, carbonate, sulfate, or phosphate, and further, it is, for example, acetate, propionate, succinate, glycolate, gluconate, lactate, malate, tartrate, citrate, ascorbate, glucronate, maleate, fumarate, pyruvate, aspartate, glutamate, benzoate, anthranylate, 4-hydroxybenzoate, phenyl acetate, mandelate, sulfonate, stearate, alginate, β-hydroxybutyrate, salicylate, galactalate, oxalate, or malonate. Or, for example, it is a metal salt containing an alkali metal or an alkaline earth metal, and further, it is a metal salt containing a transition metal such as calcium, magnesium, potassium, sodium or zinc.

In addition to the compound or a salt thereof as an active ingredient, the pharmaceutical composition of the present invention may further contain one or more pharmaceutically acceptable additives. An additive is a substance other than the active ingredient contained in pharmaceutical composition, and examples thereof include a flavoring agent, a foaming agent, a fragrance, an excipient, an isotonic agent, a surfactant, an emulsifier, a colorant, a buffer, zinc or its salt, a preservative, an antioxidant, a pH adjusting agent, a stabilizer, and a suspension agent and a solubilizing agent.

Examples of the flavoring agent include, but are not limited to, sucrose, sorbitol, citric acid, tartaric acid, malic acid and the like.

Examples of the foaming agent include, but are not limited to, sodium hydrogen carbonate and the like.

Examples of the fragrance include, but are not limited to, lemon, orange, cherry, raspberry, menthol and the like.

Examples of the excipient include, but are not limited to, lactose hydrate, crystalline cellulose, corn starch, mannitol, potato starch and the like.

Examples of the isotonic agent include, but are not limited to, sodium chloride, glucose, glycerin and the like.

Examples of the surfactant include, but are not limited to, benzalkonium chloride, benzethonium chloride, polyoxyethylene (40) monostearate (polyoxyl 40 stearate), sorbitan sesquioleate (sorbitan sesquioleate), polysorbate 20, polysolbate 80, polyoxyethylene (160) polyoxypropylene (30) glycol, sodium lauryl sulfate, glyceryl monostearate (glycerin monostearate), polyoxyethylene lauryl ether (lauromacrogol), and the like.

Examples of the emulsifier include, but are not limited to, monoglyceride, diglyceride, glycerin fatty acid ester, organic acid monoglyceride, polyglycerin fatty acid ester, propylene glycol fatty acid ester, polyglycerol condensed ricinoleic acid ester, sorbitan fatty acid ester, sucrose fatty acid ester, lecithin, enzymatically decomposed lecithin, polysorbate 20, polysolbate 80, carrageenan, guar gum, gum arabic, and the like.

Examples of the colorant include, but are not limited to, iron sesquioxide, yellow iron sesquioxide, brown iron oxide, black iron oxide, titanium oxide, edible lake pigment, riboflavin, sodium riboflavin phosphate, and the like.

Examples of the buffer include, but are not limited to, citric acid, succinic acid, fumaric acid, tartaric acid, or ascorbic acid, acetic acid or salts thereof, glutamic acid, glutamine, glycine, aspartic acid, alanine, or arginine or salts thereof, magnesium oxide, zinc oxide, magnesium hydroxide, phosphoric acid, boric acid or salts thereof, and the like.

Examples of the preservative include, but are not limited to, paraoxybenzoic acid ester (paraben), benzalkonium chloride, chlorobutanol, cresol, sodium benzoate, benzyl alcohol, sodium dehydroacetate, and the like.

Examples of the antioxidant include, but are not limited to, citric acid, sodium nitrite, ascorbic acid, sodium edetate, soy lecithin, natural vitamin E, sodium metabisulfite, dibutyl hydroxytoluene, and the like.

Examples of the pH adjusting agent include, but are not limited to, citric acid, phosphoric acid, carbonic acid, tartaric acid, fumaric acid, acetic acid or amino acid, salts thereof, and the like.

Examples of the stabilizer include, but are not limited to, sodium pyrosulfite, sodium edetate, erythorbic acid, magnesium oxide, dibutylhydroxytoluene, and the like.

Examples of the suspension agent include, but are not limited to, crystalline cellulose, sodium carmellose, xanthan gum, agar, arabia gum, tragant, sodium carboxymethyl cellulose, methyl cellulose, and the like.

Examples of the solubilizing agent include, but are not limited to, ethanol, propylene glycol, polyethylene glycol, sorbitan sesquioleate, sorbitan laurate, sorbitan palmitate, glyceryl oleate, glyceryl myristate, polyoxyethylene lauryl ether, polyoxyethylene nonylphenyl ether, glycerin, and the like.

When administered to a patient with neuronal ceroid lipofuscinosis, the pharmacological composition of the present invention can reduce lipofuscin accumulated in the patient’s body. As a result, a therapeutic effect on the patient can be achieved. In this case, a route of administration to the patient is not particularly limited, but can be appropriately selected from subcutaneous, intravenous, intramuscular, topical, oral, parenteral, transdermal, intraperitoneal, intraorbital, implantation, inhalation, arachnoid, intravesical, or intranasal administration, or the like.

A dosage form of the pharmaceutical composition of the present invention is appropriately selected according to the route of administration. The dosage form is preferably tablets, capsules, granules, powders, oral solutions, syrups, oral jelly, oral tablets, oral sprays, oral semi-solid preparations, orally disintegrating tablets, effervescent tablets, emulsions, mouthwash, injections, or inhalants. However, when orally administered to pediatric patients, from a point of view of ease of administration, the dosage form is more preferably syrups, emulsions, or orally disintegrating tablets.

When the dosage form of the pharmaceutical composition of the present invention is syrups, in addition to the active ingredient, for example, sucrose, sorbitol, or the like as a flavoring agent can be added, polyethylene glycol, sorbitan laurate, glycerin, or the like can be added as a solubilizer, and, when necessary, a preservative such as paraoxybenzoic acid ester (paraben), sodium benzoate, or benzyl alcohol can be added. In the case of a suspension syrup, in addition to the above additives, agar, arabia gum, xanthan gum, or the like can be added as a suspension agent.

When the dosage form of the pharmaceutical composition of the present invention is orally disintegrating tablets, in addition to the active ingredient, for example, sodium hydrogen carbonate or the like as a foaming agent can be added, citric acid or the like as an antioxidant can be added, and mannitol or the like as an excipient can be added.

The pharmaceutical composition of the present invention can be used in combination with enzyme replacement therapy. The enzyme replacement therapy may be effective in treating certain forms of neuronal ceroid lipofuscinosis. Even when the enzyme replacement therapy is selected for the treatment of neuronal ceroid lipofuscinosis, for a purpose of promoting or assisting a therapeutic effect of the enzyme replacement therapy on a patient, or for a purpose such as reducing the frequency of administration of enzyme preparations for supplementing enzymes, when appropriate, the pharmaceutical composition of the present invention can be used in combination with the enzyme replacement therapy.

### EXAMPLES

In the following, the present invention is described in more detail with reference to examples, using Soraprazan as an example of the compound according to the present invention. However, it is not intended that the present invention be limited to the examples.

### Evaluation Test Using Pathological Model Cells

### Example 1-1 Preparation of Pathological Model Cells

Using mammalian cells in which a causative gene of neuronal ceroid lipofuscinosis has been knocked out as pathological model cells, a test can be performed in which an effect of removing lipofuscin accumulated in the cells by Soraprazan is evaluated. For example, using PPT1 knockout (KO) HEK293T cells or PPT1 KO CHO cells, which are pathological model cells of neuronal ceroid lipofuscinosis type 1 (CLN1), a test can be performed in which an effect of removing lipofuscin accumulated in the cells by Soraprazan is evaluated. Acquisition of a PPT1 KO cell line can be performed using a conventionally known method.

### Example 1-2 Preparation of Reagents

Soraprazan is dissolved in dimethyl sulfoxide (DMSO) to a concentration of 10 mM. Subsequently, the solution is diluted with DMSO 10⁻¹ times each time in logarithmic steps to prepare DMSO solutions of Soraprazan having concentrations of 10 mM - 1 µM. For Soraprazan, commercially available products can be used, for example, Soraprazan (HY-100414, MedChemExpress) can be used. For DMSO, commercially available products can be used, for example DMSO, Anhydrous (D12345, Thermo Fisher Scientific) can be used.

### Example 1-3 Cell Culture

Knockout cells are adjusted to a constant concentration, for example, 0.2×10⁶ cells/mL, 0.3×10⁶ cells/mL, or 0.4×10⁶ cells/mL using a liquid culture medium, and this is mixed with each of the Soraprazan DMSO solutions of the concentrations prepared in Example 1-2 and 100% DMSO at a volume ratio of 99:1 to prepare 6 types of culture solutions having final Soraprazan concentrations of 100 µM - 10 nM, and 0 M. A certain amount, for example, 12 mL or 2 mL, of each of the prepared culture solutions is seeded in a flask or a 6-well tissue culture plate, respectively. The seeding is performed three times for each of the culture solutions. The flasks or the plates are placed in an incubator and cultured in a wet environment at 37° C. and 5% CO2 for a certain time period, for example, 4 days. When the cell culture is performed by shake culture, an Erlenmeyer flask is used as the flask, and it is placed in a shaker, and is shaken at a constant rotation speed of, for example, 80 rpm. As the flask, when the cell culture is performed by static culture, for example, T75 EasYFlask, TC Surface, FiterCap (Thermo Fisher Scientific) can be used, and when the cell culture is performed by shake culture, for example, Corning® 125 mL Polycarbonate Erlenmeyer Flask with Vent Cap (Corning) can be used. As the liquid culture medium, commercially available products can be used. For example, as knockout cell lines, when HEK293T cells are used, DMEM/High Glucose medium containing 10% FBS (Thermo Fisher Scientific) can be used, and when CHO cells are used, CD OptiCHOTM medium (Thermo Fisher Scientific) can be used. As the incubator, for example, Thermo ScientificTM FormaTM Steri-Cult CO2 Incubator model 3307 (Thermo Fisher Scientific) can be used, and, as the shaker, for example, MaxQTM 2000 CO2 Resistant Digital Shaker (Thermo Fisher Scientific) can be used.

### Example 1-4 Lipofuscin Fluorescence Emission Measurement

Amounts of lipofuscin accumulated in cells in the presence of sorapran at the concentrations can be compared by observing green autofluorescence of the cells using a fluorescence microscope. The cells after culturing are washed with an appropriate culture medium or buffer solution or the like, and are used for measurement. A microscopic image can be obtained for the cells cultured in the presence of Soraprazan at each of the concentrations. Measurements can be performed, for example, by measuring photoexcitation at 485 nm and fluorescence emission at 521 nm. As a fluorescence microscope, for example, EVOS FLoid Imaging System (Thermo Fisher Scientific) can be used, and in this case, wavelengths of excitation light and observation light can be respectively set to 482 nm and 532 nm. In addition, the wavelengths of the excitation light and the observation light can be changed as appropriate depending on measuring equipment or a measurement condition, and a filter or the like can also be used as appropriate.

### Example 1-5 Comparison of Measurement Results

Regarding the microscopic images obtained in Examples 1-4, using the microscopic image of the cells cultured in the absence of Soraprazan (100% DMSO added) as a reference, fluorescence intensities of the microscopic images of the cells cultured in the presence of Soraprazan at the concentrations are compared, and thereby, it can be confirmed that the amount of lipofuscin accumulated in the cells decreases according to the concentration of Soraprazan. In this way, a test can be performed in which an effect of removing lipofuscin accumulated in the cells by Soraprazan is evaluated.

### Administration Test Using Pathological Model Animals

### Example 2-1 Preparation of Experimental Animals

A test can be performed in which an effect of removing lipofuscin accumulated in animal tissues by Soraprazan is evaluated using mammals in which a causative gene of neuronal ceroid lipofuscinosis has been knocked out as pathological model animals. For example, a test can be performed in which an effect of removing lipofuscin accumulated in mouse tissues by Soraprazan is evaluated using TPP1 knockout (KO) mice, which are neuronal ceroid lipofuscinosis type 2 (CLN2) pathological model cells. Generation of the TPP1 KO mice can be performed using a conventionally known method.

### Example 2-2 Administration of Soraprazan

Soraprazan is suspended in a 0.5% aqueous solution of methyl cellulose, and the resulting suspension is orally administered to a TPP1 KO mouse administration group at an appropriate dose, for example, 0.01 mg/kg to 10 mg/kg. Or, Soraprazan is dissolved in physiological saline with or without additional solubilizers or formulation excipients, and the resulting solution is intravenously administered to a TPP1 KO mouse administration group at an appropriate dose, for example, 0.1 mg/kg to 10 mg/kg. The intravenous administration can be performed, for example, on the tail vein, the cephalic vein, or the saphenous vein. Further, a solution that does not contain Soraprazan, such as physiological saline, is administered to a negative control group at the same dose. The administration can be repeated twice daily, daily, two or four times a week for time intervals between one week and 12 months.

### Example 2-3 Measurement of Fluorescence Intensities of Tissues

After a certain time period, for example 0.25, 0.5, 1, 2, 4, 6, 8, or 24 hours after the last administration of a chronic treatment between one week and 12 months, all TPP1 KO mice are anesthetized with isoflurane and perfused with saline from the left ventricle for 4 -5 minutes for blood removal. After that, tissues (brain, heart, lungs, liver, spleen, kidneys, quadriceps, thymus, thoracic vertebrae, femur) and peripheral blood, of which fluorescence intensities are to be measured, are collected, and are stored in physiological saline to prevent drying. Fluorescence intensity measurement of lipofuscin is performed using an appropriate fluorescence intensity measuring instrument. For example, a filter set for the in vivo emission and fluorescence imaging system IVIS LuminaIII (PerkinElmer) and the fluorescent dye VivoTag750 can be used to measure the fluorescence intensity according to operating instructions.

### Example 2-4 Comparison of Measurement Results

Regarding fluorescence intensity images obtained in Example 2-3, using images of the tissues of the negative control group as references, fluorescence intensities of the tissues of the Soraprazan-administered group are compared, and thereby, it can be confirmed that Soraprazan reduces the amount of lipofuscin accumulated in each of the tissues. In this way, a dose-dependent effect of removing lipofuscin accumulated in animal tissues by Soraprazan can be evaluated. Then, based on a result of the evaluation, a suitable dosing plan can be made when administering Soraprazan as a therapeutic or prophylactic agent for neuronal ceroid lipofuscinosis.

### Industrial Applicability

According to the present invention, a therapeutic compound for neuronal ceroid lipofuscinosis can be provided.

1. A compound for treatment and/or prevention of neuronal ceroid lipofuscinosis represented by the following general formula [I], wherein
in the general formula [I], R1 is a methyl group or a hydroxymethyl group, R2 is a methyl group or a hydroxymethyl group, R3 is one of a hydroxy group, a methoxy group, an ethoxy group, an isopropoxy group, a methoxyethoxy group, a methoxypropoxy group, or an ethoxypolopoxy group, and R4 is one of a hydroxy group, a methoxy group, an ethoxy group, an isopropoxy group, a methoxyethoxy group, a methoxypropoxy group, or an ethoxypolopoxy group 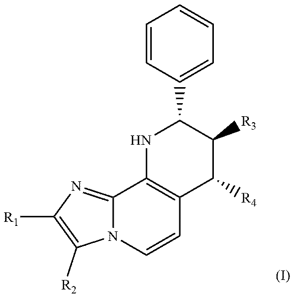
.

2. The compound according to claim 1, wherein, in the general formula [I], R1 is a methyl group.

3. The compound according to claim 1, wherein, in the general formula [I], R1 is a methyl group, and R2 is a methyl group.

4. The compound according to claim 1, wherein, in the general formula [I], R3 is a hydroxy group.

5. The compound according to claim 1, wherein, in the general formula [I], R3 is a hydroxy group, and R4 is a methoxyethoxy group.

6. The compound according to claim 1, wherein, in the general formula [I], R1 is a methyl group, R2 is a methyl group, R3 is a hydroxy group, and R4 is a methoxyethoxy group.

7. A pharmaceutical composition for treatment and/or prevention of neuronal ceroid lipofuscinosis, comprising a therapeutically effective amount of the compound according to claim 1 or a pharmaceutically acceptable salt thereof.

8. The pharmaceutical composition according to claim 7 characterized by that, when administered to a patient with neuronal ceroid lipofuscinosis, lipofuscin accumulated in the patient’s body is reduced.

9. The pharmaceutical composition according to claim 8, wherein the administration is subcutaneous, intravenous, intramuscular, parenteral, local, oral, transdermal, intraperitoneal, intraorbital, implantation, inhalation, intraarachnoid, intravesical, or intranasal administration.

10. The pharmaceutical composition according to claim 7, being in a dosage form selected from a group consisting of tablets, capsules, granules, powders, oral solutions, syrups, oral jelly, oral tablets, oral sprays, oral semi-solid preparations, orally disintegrating tablets, parenteral emulsions, parenteral suspensions, parenteral solutions, effervescent tablets, mouthwash, injections, and inhalants.

11. The pharmaceutical composition according to claim 7, further comprising at least one pharmaceutically acceptable additive.

12. The pharmaceutical composition according to claim 11, wherein the at least one additive is selected from a group consisting of a flavoring agent, a foaming agent, a fragrance, an excipient, an isotonic agent, a surfactant, an emulsifier, a colorant, a buffer, zinc or its salt, a preservative, an antioxidant, a pH adjusting agent, a stabilizer, and a suspension agent or a solubilizing agent.

13. The pharmacological composition according to claim 7, being used together with enzyme replacement therapy for a patient with neuronal ceroid lipofuscinosis.
